(19) United States
(12) Patent Application Publication
(10) Pub. No.: US 2015/0150355 A1
(43) Pub. Date: Jun. 4, 2015

(54) Anti-Wrinkle Miracle Devise

(71) Applicant: Edwin Crespo, Juncos, PR (US)

(72) Inventor: Edwin Crespo, Juncos, PR (US)

(21) Appl. No.: 14/094,065

(22) Filed: Dec. 2, 2013

(51) Int. Cl. A45D 44/22; A61F 9/04

(52) U.S. Cl. CPC A45D 44/22; A61F 9/04

(57) ABSTRACT

A piece of non-allergenic wire such as 925 silver with ⅜×⅜ in thin flat square swivel plates, each with Hypoallergenic double-sided tape on the outer side. The wearer fixes one plate just above one eyebrow and the other end under the eye, both to the corner of the eye area; the purpose is that when at bedtime the user is trying to fall asleep she or he unconsciously makes grimaces, or, by pressure of the pillow, creates temporary and unwelcomed wrinkles on the eye area, which, with time become permanent wrinkles, with this invention placed on such area, both, upper and lower eye areas are prevented to move closer together, thus, the appearance of temporary and permanent wrinkles are greatly reduced.

A piece of wire (1, FIG. 1) specially designed to be attached over one or both eyes, one end is fixed by means of non-allergenic adhesive tape just above one eyebrow and the other end just under one's person eye. This practice prevents areas between both ends move closer to one another due to the opposite force exerted by the wire and the plates (2, FIG. 1) when the person has the eye area stressed, makes grimaces or by pressure over the pillow.

Most people cannot stay completely relaxed specially on the eye area while sleeping due to the stress the person experiences during daily living or the eyes may be stressed due to contact with a pillow or mattress, this stress over the skin produces wrinkles to the eye area making humans look older; the present invention helps prevent wrinkles on that area.

Each piece of wire is provided with two swivel flat members to adapt the anti-wrinkle devise to the contour of the user's face and where the pieces of adhesive tape are positioned.

This invention is preferably built in 925 silver, gold, plastic, or any other non-allergenic material. The wire pieces do not necessarily have to be round; they could have different shapes, such as flat, square, etcetera.

The pieces of wire are preferably slightly bent outwards to avoid contact with eyelashes when blinking.

The wires, between 0.01 and 0.03 ml diameter and from 2-4 inches long allow for different stiffness effects on the skin, especially when the user smiles, the wires bend slightly. It is possible and right to smile while wearing the devises.

When over the eyes and looking somewhere the wires are almost invisible, the same way it happens while wearing glasses, when the frame is thin the user almost do not see it.

By attaching only one wire to an eye, both eyes are automatically trained to get relaxed because the pressure exerted over the skin signals the user that he or she is usually stressed on the area of both eyes.

Exactly the way any person gets habituated to wear belts, brassieres, contact lenses, eyeglasses, false teeth, earrings and so on, with practice he or she will soon get familiar with this practice.

Another version is the Anti-Wrinkle Devise with two wires, the one previously described plus an extra one with an extra swivel member to be stickled just by the side of the nose to protect that area from wrinkles in the case the user deems it is needed.

Still another benefit of this invention is that the user will get a better night's sleep by improving her overall skills in stress relaxation techniques.

To familiarize easier it is suggested to start using this devise during daytime either on one eye or both.

When the user is preparing to go to sleep, the person may, mentally program himself or herself to meditate and relax, not only the face's muscles but of the whole body.

After several days of using a single unit the user may try on only one side of the face or both simultaneously with the double anti-wrinkle (3, FIG. 2) or with the Glasses, which hold the concealed Anti-Wrinkle devise (4, FIG. 3) in such a way that are difficult to be seen during regular use.

It is easily possible by the user to blink while using this devise without any annoyance because there is an adequate distance between the eyes and the wires.

Another extraordinary version of this invention is an especially designed eyeglasses frame that is able to hold the non-visible adhesive tape to be secured to the skin and so attaining exactly the same effect previously explained, which is the essence of this invention, the difference with the glasses is that they may be worn while being awake on almost any occasion. The non-allergenic adhesive means are non-visible from the outside.

The Anti-Wrinkle Miracle Devise may include only one set of wire member or several, one pair of eyeglasses or several or any combination to suit preferences such as price, effects, color or type of wrinkle's treatment depending on particular personal needs.

### The accompanying four drawings show as follows:

FIG. 1—A user with one single unit of the invention.

2, FIG. 1—Exploded view of how a wire is fixed to the plate.

FIG. 2—a person wearing a double function anti-wrinkle devise.

FIG. 3 show how one single unit of the invention looks attached on the inner side of a pair of glasses.

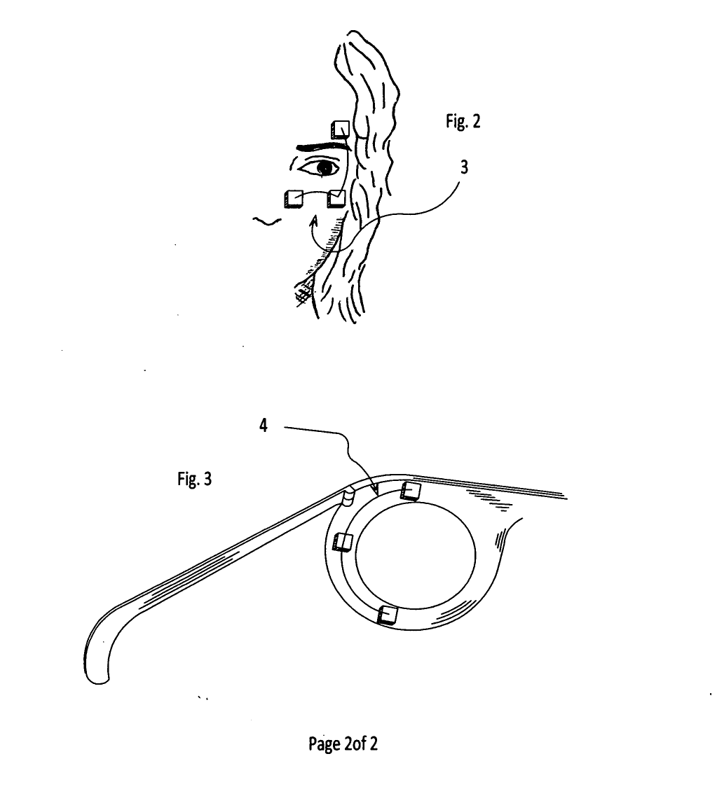

What is claimed is:

1. A piece of wire between 0.01 and 0.03 ml in diameter and from 2-4 in long, holding flat square swivel plates made in hypoallergenic metal such as 925 silver, ⅜×⅜ in and from 0.005-0.015 ml thick which are secured to each end by means of loops on both components, such sheets have hipoallergenic adhesive tapes on their outer ends to be stickled to the upper and lower human outside eye area with the purpose of preventing both areas from getting closer together, thus, causing wrinkles when the person makes voluntary or not face movements.

2. As in claim No. 1 with the devise concealed in the inside part of specially designed pair of glasses, placed right behind the frame.

3. As in claim No. 2, further including one attached single platted gadget extending horizontally from the outer and lower eye area to the cheek area center of the nose.
